(19) United States
(12) Patent Application Publication
(10) Pub. No.: US 2009/0035797 A1
(43) Pub. Date: Feb. 5, 2009

(54) Detection of Disease Associated Proteolysis

(76) Inventors: William S. Hancock, Brookline, MA (US); Haven Baker, Roslindale, MA (US); Marina Hincapie, Framingham, MA (US); Xiaoyang Zheng, Malden, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/665,403

(22) PCT Filed: Oct. 17, 2005
(86) PCT No.: PCT/US05/37024

§ 371 Date: Mar. 5, 2008

Related U.S. Application Data
(60) Provisional application No. 60/619,162, filed on Oct. 15, 2004.

(51) Int. Cl. C12Q 1/37; C12Q 1/02; C07K 1/20; G01N 33/48; C12Q 1/00

(52) U.S. Cl. 435/23; 435/4; 435/29; 530/344; 250/282

(57) ABSTRACT

Described herein are methods and techniques to study the “degradome”. The degradome of a specific protease is the complete product of the natural substrate repertoire of that enzyme in a cell, tissue or organism. The complete set of proteases that are expressed at a particular moment or circumstance by a cell, tissue or organism produces the collective degradome. Included in the methods described herein are approaches that allow the direct identification and characterization of degradome peptides from approx. 400 to approx. 12,000 Da. The methods of the invention avoid the inherent problems of studying the peptidome by focusing on specific or unique proteolytic cleavages that occur as a result of endogenous protease activity induced by specific diseases. Once characterized, the presence of, or change in level of, specific peptides of the degradome can be used, e.g., to identify specific peptides having elevated levels compared to a reference normal/or to correlate identified peptides with specific proteins and/or to identify protein fragmentation patterns (e.g., peptide ladders) and the specific protease(s) that brought them about and then correlate this information with the presence or absense of a specific disease or condition. Thus, the methods of the invention can be used, for example, to identify new diagnostic markers and/or therapeutic targets, as specific clinical diagnostic methods for individual patients and as methods of monitoring the progress of a therapeutic regimen for the treatment of a patient.

### CROSS REFERENCE TO RELATED APPLICATIONS

This application claims the priority of U.S. Provisional Application No. 60/619,162 filed Oct. 15, 2004 entitled, USING LIQUID CHROMOTOGRAPHY-MASS SPECTROMETRY TO ANALYZE NATIVE HUMAN SERUM PEPTIDES, the whole of which is hereby incorporated by reference herein.

### STATEMENT REGARDING FEDERALLY SPONSORED RESEARCH OR DEVELOPMENT

N/A

### BACKGROUND OF THE INVENTION

Human serum is one of the most informative of bodily fluids as it provides the major link among many human organs, tissues and cells. It has been estimated that the serum proteome consists of tens of thousands of proteins/peptides, with a large concentration range from 35-50×10⁹ pg/mL to 0-5 pg/mL.¹ This proteome includes 22 abundant and well-characterized proteins, which represent 99% of the mass of the proteinaceous content of human serum.²

Advances in proteomics are continuing to expand the ability of investigators to analyze the serum proteome. In recent years it has been realized that in addition to the circulating proteins, human serum also contains a large number of peptides, frequently called the “peptidome.” Many of these peptides are believed to be fragments of larger proteins that have been at least partially degraded by various enzymes such as metalloproteases. However, identifying these proteins of origin from a small amount of serum/plasma is difficult due to the complexity of the sample, the low levels of these peptides and the difficulties in getting a protein identification from a single peptide.

To date, researchers have used different approaches to characterize the low molecular weight (LMW) protein or peptide species in serum. In the past five years, Richter, Jurgens and colleagues have published a number of papers describing fractionation of both hemofiltrate and serum and use of MALDI-TOF and electrospray instruments for analysis.^{4,5} More recently, different approaches using ultrafiltration to extract the peptides from serum have been published. Tirumalai et al. adopted 2D-LC-MS/MS and identified 340 proteins.² Forssmann et al. used MALDI-MS and obtained a differential peptide display.²³ Zhou et al. used immunoaffinity or ligand affinity chromatography and 1D-LC-MS/MS technologies and identified 210 proteins.⁶ Harper et al. analyzed LMW proteins/peptides with isoelectric focusing and RPLC-MS/MS and identified 262 proteins.⁷ However, improved methods of analyzing the peptidome would be desirable.

### BRIEF SUMMARY OF THE INVENTION

The invention is directed to methods and techniques to study the “degradome.” The degradome of a specific protease is the complete product of the natural substrate repetoire of that enzyme in a cell, tissue or organism. The complete set of proteases that are expressed at a particular moment or circumstance by a cell, tissue or organism produces the collective degradome. Included in the methods described herein are approaches that allow the direct identification and characterization of degradome peptides from approx. 400 to approx. 12,000 Da. The methods of the invention avoid the inherent problems of studying the peptidome by focusing on specific or unique proteolytic cleavages that occur as a result of endogenous protease activity induced by specific diseases. Once characterized, the presence of, or change in level of, specific peptides of the degradome can be used, e.g., to identify specific peptides having elevated levels compared to a reference normal and/or to correlate identified peptides with specific proteins and/or to identify protein fragmentation patterns (e.g., peptide ladders) and the specific protease(s) that brought them about and then correlate this information with the presence or absense of a specific disease or condition. Thus, the methods of the invention can be used, for example, to identify new diagnostic markers and/or therapeutic targets, as specific clinical diagnostic methods for individual patients and as methods of monitoring the progress of a therapeutic regimen for the treatment of a patient. The methods and techniques described herein enable the identification of protein substrates, which enable the discovery of the molecular basis of biological pathways or processes associated with proteolysis, including for example, cancer, inflammation, muscular, neurodegenerative and autoimmune diseases.

Cleavage of target protein substrates can directly or indirectly affect the activity or function of numerous proteins, enzymes and receptors, and other proteins within a biological pathway. This can lead to a cascade of events that may trigger intracellular signaling or may lead to changes in various cell activities, including, for example, cell spreading, migration, cell-cell adhesion, ectodomain shedding and cell death.

Thus, in general, the invention is directed to a method for detecting disease associated proteolysis comprising the steps of providing a sample of bodily fluid from a mammal; isolating from said bodily fluid sample all low molecular weight peptides having a molecular weight of not more than approximately 12,000 Daltons; directly detecting and determining the amino acid sequences of said isolated peptides by ionization mass spectrometry; and relating the determined sequence information to a reference standard. Preferably, the sample is a bodily fluid selected from the group consisting of blood (e.g., plasma or serum), saliva, urine, nipple aspirate, ductal lavage, sweat or perspiration, tumor exudates, joint fluid (e.g. synovial fluid), inflammation fluid, tears, semen and vaginal secretions. The step of isolating can include one or more methods selected from the group consisting of reverse phase chromatography, ultrafiltration and bulk electrophoresis, and the step of directly detecting can be carried out by electrospray ionization mass spectrometry, preferably using an ion trap mass spectrometer, more preferably, a hybrid ion trap mass spectrometer coupled to a quadrapole, time-of-flight mass spectrometer, and most preferably, a reversed phase-high performance liquid chromatography system connected to a nanospray ionization hybrid ion trap-fourier transform mass spectrometer. The relating step preferably includes one or more methods selected from the group consisting of identifying specific peptides having elevated levels compared to a reference normal, correlating peptides with specific proteins, and identifying protein fragmentation cleavage sites and relating that cleavage site to the activity of a specific endogenous propeolytic enzyme. The method can further comprise, prior to the step of directly detecting, further fractionating said isolated peptides by removing all peptides having a molecular weight of less than approximately 3,000-4,000 Daltons.

In another aspect, the invention is directed to a method of identifying a potential biomarker for a disease or pathological condition, the method including the steps of obtaining a plurality of biological samples from a plurality of donors, wherein said plurality of donors comprise healthy individuals and patients at high risk for and/or having said disease or pathological condition; isolating from the plurality of bodily fluid samples all low molecular weight peptides having a molecular weight of not more than approximately 12,000 Daltons; directly detecting and determining the amino acid sequences of the isolated peptides by mass spectrometry; determining a profile of low molecular weight peptide abundance in each of the samples; comparing the abundance profiles of the patients at high risk for and/or having the disease or pathological condition with the abundance profiles of the healthy individuals; and identifying any low molecular weight peptides that are present in the abundance profiles of the patients at high risk for and/or having the disease or pathological condition but not in, or at a reduced level in, the abundance profiles of the healthy individuals, wherein any low molecular weight peptide so identified is a potential biomarker for the disease or pathological condition. Preferably, this method further comprises repeating the method steps so as to identify a plurality of potential biomarkers for the disease or pathological condition and the plurality of potential biomarkers for the disease or pathological condition includes a panel of potential biomarkers.

In yet another aspect, the invention is directed to a method of separating small peptides from large peptides and proteins comprising providing a column comprising polymeric, silica, zirconia reversed phase chromatographic particles equilibrated to retain hydrophobic compounds and not to retain salts and other hydrophilic compounds; applying a sample comprising a mixture of proteins and peptides to said column; washing hydrophilic compounds through said column; washing said column to remove mildly hydrophobic, small peptides from said column; washing said column to remove larger, more hydrophobic peptides away from proteins from said column; and washing said column to separate proteins from mildly and very hydrophobic peptides. The method can comprise further providing a second column comprising a polymeric, silica, zirconia reversed phase chromatographic resin equilibrated to retain small, hydrophilic compounds eluted from said first column.

### BRIEF DESCRIPTION OF THE DRAWINGS

Other features and advantages of the invention will be apparent from the following description of the preferred embodiments thereof and from the claims, taken in conjunction with the accompanying drawings, in which:

FIGS. 1A and 1B show SDS-PAGE analysis of human serum ultrafiltrate. FIG. 1A shows the staining of a 1D SDS-PAGE gel with the Coomassie® blue. FIG. 1B shows the same gel, destained and then stained with silver. Lane 1 is 12 μL of SeeBlue™ pre-stained standards; lane 2 is 9 μg of protein from 1.5 μL of human serum diluted into 9 μL; lane 3 is 37 μg of sample, ultrafiltered from 57 μL of human serum, which has been precipitated in solvents and reconstituted; lane 4 is the same human serum filtrate, concentrated with a speed-vac and reconstituted. The molecular weights of the pre-stained standards are shown on the left of the gel. Bands in lanes 3 and 4 are defined from top to bottom as a, b, c, d, and e, respectively;

FIG. 2 shows RP-LC/MS base peak ion chromatograms from triplicate analyses of the 10,000 Da human serum filtrate. The sample (10 μL) was loaded for 60 minutes with a flow rate of 200 nL/min in 2% mobile phase B (0.1% formic acid, 100% acetonitrile). Peptides were eluted with a 60 minute gradient (2% B to 40% B in 35 min, 40% B to 60% B in 20 min) and washed in 80% B for 5 min;

FIGS. 3A and 3B show full MS scan and MS/MS spectrum, respectively, of peptide A.SEGGFTATGQR.Q from extracellular matrix protein 1. FIG. 3A: Full MS scan occurred when the solvent composition was at 15.5% B; the insert is a magnified view of the precursor ion showing a MW of 555.7634 Da for this peptide. FIG. 3B: MS/MS spectrum of this peptide. This is an example where the mass accuracy is less than 2 ppm;

FIGS. 4A and 4B show Venn diagrams from triplicate analyses of human serum ultrafiltrate. FIG. 4A illustrates the number of overlapping protein identifications from repeat analysis; 75 proteins were unique to run 1, 22 were present in both runs 1 and 2, 17 were present in both runs 1 and 3, and 55 proteins were present in all three runs. FIG. 4B illustrates the number of overlapping peptide identifications from the same analysis. Filter criteria: Xcorr(1+,2+,3+)=1.8, 2.5, 3.5;

FIG. 5 shows distribution of peptides by molecular weight. The 337 unique peptides listed in Table 2 were observed in at least two runs and have both an Xcorr(1+,2+,3+)≧1.8,2.5,3.5 and at least 2 ppm mass accuracy;

FIG. 6 shows the location of identified fibrinogen α/α-E chain peptides. The boxes refer to the sequence of the peptide ladders that were observed in the sample. The shaded box indicates the predominant cleavage sites;

FIG. 7 shows tissue specificity of the 61 proteins whose peptide(s) were identified in at least two analyses, with a mass accuracy of at least 2ppm. Tissue specificity was obtained from SwissProt (http://au.expasy.org/sprot/);

FIG. 8 shows cellular component information for the 61 proteins of FIG. 7. The cellular component was obtained from LocusLink (http://www.ncbi.nlm.nih.gov/projects/LocusLink/);

FIG. 9 shows the molecular function of the 61 proteins of FIG. 7. Molecular function information was obtained from LocusLink (http://www.ncbi.nlm.nih.gov/projects/LocusLink/);

FIG. 10 is a graph showing material eluted from a C18 reverse phase column over time versus percent hydrophobic phase; and

FIG. 11 is a schematic diagram of a two-column set-up for practicing the method of the invention.

### DETAILED DESCRIPTION OF THE INVENTION

Advances in proteomics are continuing to expand the ability of investigators and clinicians to analyze the serum proteome. In recent years, it has been realized that in addition to the circulating proteins, human serum also contains a large number of peptides. Many of these peptides are believed to be fragments from proteolytic processing by various enzymes, such as metalloproteases. Identifying these peptides from a small amount of serum or plasma is difficult due to the complexity of the sample, the low levels of these peptides, and the difficulties in getting a protein identification from a single peptide.

In the methods described herein, the protein/peptide population is depleted of high molecular weight material using, e.g., reverse phase chromatography, and/or centrifugal ultrafiltration and/or bulk electrophoresis. Unlike in previous protocols using ultrafiltration, the isolated low molecular weight peptides are not digested, e.g., with trypsin, prior to analysis. This means that what is identified is the peptides that are produced endogenously. The fraction depleted of high molecular weight material was then concentrated and analyzed by mass spectrometry.

To obtain additional data, the sample fraction depleted of high molecular weight material may be additionally depleted of the lowest molecular weight material (e.g., from 400-3-4,000 Da), and the “mid-range” low molecular weight material (3-4,000-12,000 Da) can be analyzed separately. Peptides in the range of 400-4000 Da can be sequenced by using conventional ion trap mass spectrometers (e.g., LTQ-MS from Thermo Electron (San Jose, Calif., USA). However, confident identifications of these peptides requires a high mass accuracy (<5 ppm) instrument such as the hybrid ion trap-fourier transform mass spectrometer (LTQ-FTMS). High mass accuracy combined with a peptide sequence allows for the identification of a protein substrate from a single peptide.

Sequencing of the mid-range peptides (4,000-12,000 Da) requires the LTQ-FT hybrid-ion trap, or another mass spectrometer system that combines sequencing capabilities with mass analyzer having a resolution of at least 25,000. Preferably, the mass spectrometer system includes a mass spectrometer having a mass resolution of at least 50,000 and may include separate mass spectrometer instruments having the indicated properties. Currently, the best solution for identifying individual mid-range peptides in the degradome is a hybrid instrument, the LTQ-FTMS, connected to a reversed phase-high performance liquid chromatography system with a nanospray ionization source. The mass accuracy of this instrument allows the user to identify multiply charged peptides and determine their charge state, thus confidently identifying the protein precursors with the use of single peptides. As described herein, peptides in the molecular weight range of 400-4000 Da and/or those having a molecular weight of 4,000-12,000 Da can be used for the purposes of identification.

The utility of the hybrid ion trap (LTQ-FTMS) was demonstrated by the identification and characterization of over 300 unique peptides per serum sample with 2 ppm or better mass accuracy. With confident identifications, the origin and function of native serum peptides can be determined. Interestingly, over 34 peptide ladders were observed from over 17 serum proteins. This indicates that a cascade of proteolytic processes affects the serum peptidome. To examine whether this result was an artifact of serum, matched plasma and serum samples were analyzed, with similar peptide ladders found in each.

The progenitor proteins identified herein are of clinical interest for disease monitoring (see, e.g., Table 2 and proteins listed therein, e.g., apolipoproteins (cardiovascular disease), complement factors and prothrombin (blood clotting disorders), thymosin (immune system), plectin (bullous pemphigoid), extracellular matrix protein (cancer), ephrin receptor (cancer), insulin-like growth factor binding protein (diabetes), and vitamin D-binding protein (cancer).

Many diseases, particularly cancer and inflammation-based disorders are associated with altered patterns of proteolysis, which can be associated with changes, e.g., in the regulation of metalloproteases and other enzymes.²⁰ Table 6 shows the specificity of proteases responsible for the generation of the peptides listed in Table 3. The table shows the number of observed enzymatic cleavages between two specific amino acids for a given serum sample. From a table such as this, one can locate cleavage patterns that are indicative of specific proteases. It can be seen that enzymes with trypsin-like specificity represent only part of the observed cleavage sites. If one presumes that the observed peptides were produced by endoproteases, which cleave on the C-terminal side of amino acid residues, then the most common N-termini of the observed peptides were glycine (57 times), serine (39) and aspartic acid (36), while lysine (59) and arginine (76) predominate following the C-terminus. Exoprotease cleavages have also been observed.

TABLE 6
Examination of the N- and C-terminal amino acids of all unique peptides
to indicate proteases involved in serum peptide generation.
All peptides were observed in at least two runs and have both an
Xcorr 1+, 2+, 3+) ≧ 1.8, 2.5, 3.5 and at least 2 ppm mass accuracy.

| | | N-terminus of Peptide | | | |
|---|---|---|---|---|---|
| | | C-terminal | | C-terminus of Peptide | |
| | | amino acid | N-terminal | C-terminal | N terminal |
| Grouped by | | of the | amino acid of | amino acid | amino acid of |
| Protease | | preceding | identified | of identified | the following |
| Characteristics | Amino Acid | peptide | peptide | peptide | peptide |
| Hydrophilic | Lysine (K) | 45 | 13 | 4 | 59 |
| Basic | Arginine (R) | 68 | 4 | 26 | 76 |
| | Histidine (H) | 5 | 14 | 26 | 3 |
| Hydrophilic | Aspartic Acid (D) | 8 | 36 | 3 | 6 |
| Acidic | Glutamic Acid (E) | 20 | 31 | 13 | 8 |
| Hydrophilic | Glycine (G) | 31 | 57 | 37 | 28 |
| Polar | Serine (S) | 25 | 39 | 25 | 29 |
| | Threonine (T) | 6 | 36 | 28 | 13 |
| | Cysteine (C) | 0 | 0 | 0 | 1 |
| | Asparagine (N) | 3 | 9 | 15 | 11 |
| | Glutamine (Q) | 9 | 9 | 19 | 3 |
| | Tyrosine (Y) | 8 | 3 | 10 | 4 |
| Hydrophobic | Alanine (A) | 29 | 18 | 20 | 23 |
| Non-Polar | Valine (V) | 4 | 13 | 19 | 5 |
| | Leucine (L) | 25 | 15 | 25 | 11 |
| | Isoleucine (I) | 5 | 8 | 2 | 10 |
| | Proline (P) | 20 | 12 | 13 | 5 |
| | Methionine (M) | 7 | 1 | 0 | 2 |
| | Phenylalanine (F) | 10 | 5 | 16 | 7 |
| | Tryptophan (W) | 2 | 0 | 3 | 2 |

Nomenclature in this tables is as follows:

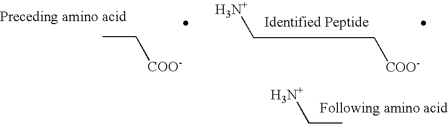

Analysis of changes in cleavage patterns such as these will be of value in linking altered proteolysis with disease. For example, in a clinical assay, any specific type of proteolytic cleavage, e.g., by an aspartic, cysteine, metallo, serine/threonine or other type of protease, can be determined as described herein by identifying the LMW peptides by mass spectrometry, and any changes in the observed cleavage patterns over time can be followed. As another example, a specific peptide determined to be a “biomarker” for the specific type of proteolysis can be detected as either in high or low abundance compared to a reference standard, and the determined value can be correlated with the extent of the related disease or condition. The protein substrate of the indicated proteolytic enzyme can also be useful as a biomarker. Biomarkers of this type can originate from any tissue or cellular compartment or can be generated by digestion of secreted and surface proteins.

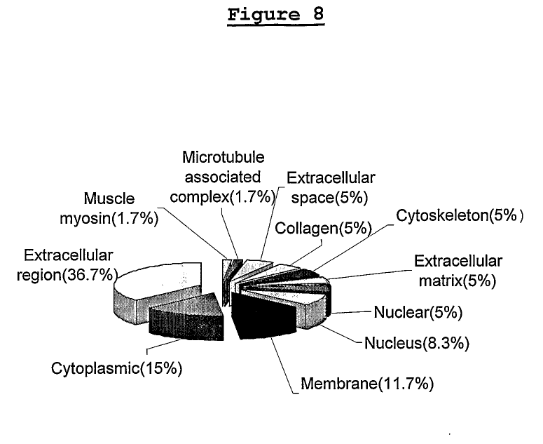

For example, FIG. 7 lists the tissue of origin (as given in SwissProt) for 61 proteins based on peptide identifications herein with 2 ppm or better mass accuracy. As could be expected, the classification is dominated by those tissues responsible for the majority of secreted proteins in blood such as liver (23.3%), blood (16.6%), brain (13.3%), heart and muscle (both 10%). We also examined the same set of peptide identifications for cellular compartment of origin in expectation that the major fraction of the peptidome would be generated by digestion of secreted and surface proteins. The results are given in FIG. 8 and show that extracellular region is indeed the major compartment (36.7%), with smaller contributions from cytoplasmic (15%) and membrane (11.7%) sites.

Another way of assessing the origin of the degradome is to examine the gene ontology of the set of high confidence progenitor proteins using publicly available software (FuncAssociate http://llama.med.harvard.edu/cgi/func/funcassociate).²¹ Table 7 shows those attributes with an adjusted P-value of less than 0.001, and the most highly ranked categories include extracellular, organismal physiological process, endopeptidase and protease inhibitor, blood coagulation and clotting, acute-phase response and lipid transport and metabolism.

TABLE 7
Sixty-one proteins identified according to the method of the invention
grouped by function using FuncAssociate software
(http://llama.med.harvard.edu/cgi/func/funcassociate).

| | Number | | | | | | |
|---|---|---|---|---|---|---|---|
| | of Genes | | | | | | |
| | with this | | | | | Gene | |
| Rank | attribute | X^{a} | LOD^{b} | P Value | P-adj^{c} | Ontology | Class of Proteins |
| 1 | 30 | 1454 | 1.199 | 4.3e−21 | <0.001 | 0005576 | extracellular |
| 2 | 27 | 2208 | 0.914 | 3.8e−13 | <0.001 | 0050874 | organismal physiological |
| | | | | | | | process |
| 3 | 12 | 488 | 1.104 | 2.5e−09 | <0.001 | 0009613 | response to pest, pathogen or |
| | | | | | | | parasite/response to |
| | | | | | | | pest/pathogen/parasite |
| 4 | 9 | 216 | 1.325 | 3.2e−09 | <0.001 | 0004866 | endopeptidase inhibitor |
| | | | | | | | activity/endoproteinase |
| | | | | | | | inhibitor/proteinase inhibitor |
| 5 | 9 | 217 | 1.323 | 3.4e−09 | <0.001 | 0030414 | Protease inhibitor |
| | | | | | | | activity/peptidase inhibitor |
| 6 | 7 | 93 | 1.588 | 3.4e−09 | <0.001 | 0007596 | Blood coagulation/blood |
| | | | | | | | clotting |
| 7 | 12 | 503 | 1.090 | 3.5e−09 | <0.001 | 0043207 | response to external biotic |
| | | | | | | | stimulus |
| 8 | 5 | 25 | 2.064 | 4.2e−09 | <0.001 | 0006953 | acute-phase response |
| 9 | 7 | 100 | 1.554 | 5.6e−09 | <0.001 | 0007599 | hemostasis |
| 10 | 7 | 109 | 1.514 | 1e−08 | <0.001 | 0050878 | regulation of body fluids |
| 11 | 6 | 65 | 1.680 | 1.4e−08 | <0.001 | 0006869 | lipid transport |
| 12 | 7 | 117 | 1.481 | 1.7e−08 | <0.001 | 0050817 | coagulation/clotting |
| 13 | 6 | 69 | 1.652 | 2e−08 | <0.001 | 0005319 | lipid transporter |
| | | | | | | | activity/lipophorin |
| 14 | 17 | 1367 | 0.828 | 2.3e−08 | <0.001 | 0009605 | response to external stimulus |
| 15 | 5 | 39 | 1.837 | 4.4e−08 | <0.001 | 0042157 | lipoprotein metabolism |
| 16 | 23 | 2692 | 0.699 | 4.6e−08 | <0.001 | 0005515 | protein binding |
| 17 | 9 | 299 | 1.177 | 5.4e−08 | <0.001 | 0005615 | extracellular space |
| 18 | 14 | 995 | 0.863 | 1.2e−07 | <0.001 | 0006950 | response to stress |
| 19 | 9 | 330 | 1.132 | 1.2e−07 | <0.001 | 0004857 | enzyme inhibitor activity |
| 20 | 6 | 106 | 1.452 | 2.6e−07 | <0.001 | 0008015 | circulation |
| 21 | 8 | 287 | 1.137 | 5.6e−07 | <0.001 | 0009611 | response to wounding |
| 22 | 4 | 27 | 1.909 | 5.9e−07 | <0.001 | 0006958 | complement activation, |
| | | | | | | | classical pathway |
| 23 | 13 | 989 | 0.825 | 7.7e−07 | <0.001 | 0006955 | immune response |
| 24 | 14 | 1236 | 0.763 | 1.6e−06 | <0.001 | 0009607 | response to biotic stimulus |
| 25 | 13 | 1082 | 0.784 | 2.1e−06 | 0.001 | 0006952 | defense response/defense |
| | | | | | | | response |
| 26 | 4 | 37 | 1.755 | 2.2e−06 | 0.001 | 0006956 | complement activation |

^{a}Total number of genes with this attribute.
^{b}The natural log of the odds ratio,
^{c}adjusted P-value: fraction (as a %) of 1000 null-hypothesis simulations having attributes with this or a lower P value.²¹

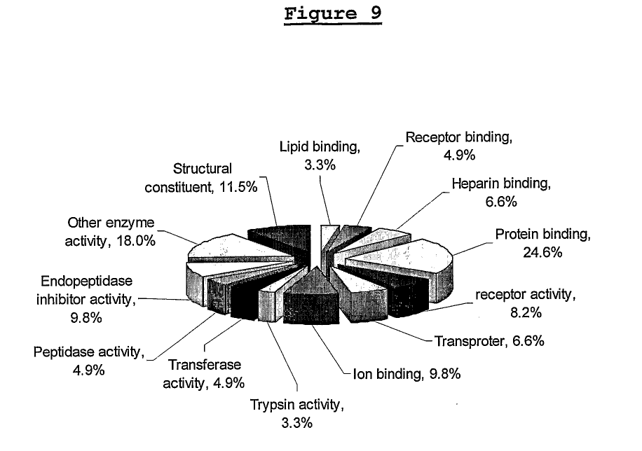

FIG. 9 shows the types of biological activities associated with these 61 identified proteins, and a large proportion of the activities are associated with proteolysis, such as endopeptidase, peptidase activity, trypsin activity and other enzyme activity (total of 31%). Other interesting categories were receptor activity and receptor binding protein which together totaled 13%. These results again suggest that analysis of the peptidome will be valuable for the study of changes in disease associated proteolysis of surface receptors, secreted and shed proteins during processes such as apoptosis.

Thus, in general, the methods described herein can be practiced on any type of patent sample, including, without limitation, samples of bodily fluid taken from blood (e.g., plasma or serum), saliva, urine, nipple aspirate, ductal lavage, sweat or perspiration, tumor exudates, joint fluid (e.g., synovial fluid), inflammation fluid, tears, semen and vaginal secretions. Cells, tissues or organelles can also be sampled successfully if a protease cocktail is added immediately upon cell lysis.

The identified peptides can be used for the direct identification of precursor proteins and involved proteolytic enzymes, as described, or, e.g., pooled information from a number of patients can be used for clinical development of mass spectrometry-based diagnostics assays and for biomarker identification, particularly for the identification of multiple biomarkers that can be used simultaneously in panels. The identified peptides can also be used 1) to monitor the therapeutic response to specific protease inhibitors; 2) for selection and screening of drug candidates in preclinical studies; 3) for patient selection and response in clinical drug development; 4) for rational design of peptide inhibitors for specific proteases; 5) for the identification of novel proteases; and 6) to gain knowledge about the mechanism of disease etiology. The methods described herein can also be used to detect aberrant protein expression or function due to genetic mutations that may result in proteolytic degradation and subsequent peptide generation.

In an alternative use, the method of the invention can be used to explore the set of substrates for a selected protease, e.g., a metalloprotease, in a sample. A plasma or cell extract could be incubated with the protease and the cleavage points could be determined. This information would allow one to look for the same patterns in a specific disease and use the observation of an associated panel of peptides in the sample to show the protease was upregulated in that disease. Furthermore, information concerning the peptides of the degradome can be used to look at the pathology of a diseased sample by determining the cleavage patterns either in the tissue or in a fluid and then using that information to identify the protease. One can then image the tissue with an antibody to the peptide and localise the enzyme. Additionally, the method of the invention can be used to identify a set of proteases up or down-regulated in a specific disease or condition and then the proteases can be prepared by cloning or synthesis and attached to a chip as a protease array. A disease sample can then be added to the array along with fluorescent peptide substrates. If the peptide is in the sample it will compete with the fluorogenic peptide and a decrease in intensity will be observed.

The following examples are presented to illustrate the advantages of the present invention and to assist one of ordinary skill in making and using the same. These examples are not intended in any way otherwise to limit the scope of the disclosure.

### Materials and Methods

### Materials

Matched human serum and plasma sets were purchased from Bioreclamation, Inc. (Hicksville, N.Y.). Vivaspin® 4 centrifugal concentrators with a molecular weight cut off (MWCO) of 10,000 Da were obtained from ISC Bioexpress (Kaysville, Utah). Microcon YM-10 Centrifugal Filter Unit and Ziptip® with 0.6 μL C18 resin were purchased from Millipore (Bedford, Mass.).

### Ultrafiltration

Centrifugal concentrators were pre-rinsed with deionized water several times to minimize contamination of their membranes. For each sample, human serum (or plasma) was diluted in distilled and deionized water that contained 10-30% (v/v) acetonitrile in a ratio of 1:3 and then incubated at room temperature for 30 min. The diluted serum (or plasma) was transferred to a centrifugal filter with a MWCO of 10,000 Da and spun in a centrifuge to deplete the high molecular weight proteins (HMW). At the end of the process, approximately 80% of diluted filtrate was collected and peptides desalted using either Ziptip® with 0.6 μL C18 resin or reverse phase chromatography using a C18 column. (see below)

Alternatively, the filtrate can be subjected to a second round of ultrafiltration to separate the low mass range peptides (400-3-4,000) from the larger (3-4,000-12,000) peptides. The filtrate obtained from the 10,000 MWCO filter is apply to a centrifugal filter with a MWCO of 5,000 Da. And spin in a centrifuge. The small range peptides are collected in the filtrate, while the larger peptides (3-4,000-12,000 Da) are collected from the retained fraction. Both fractions are desalted using either Ziptip® with 0.6 μL C18 resin or reverse phase chromatography using a C18 column. (see below)

### Protein Precipitation

The filtrate (200 μL) was first diluted with seven volumes (1400 μL) of cold acetonitrile (stored at −20° C. for at least 2 hours). After that, the mixture was vortexed vigorously for 5 seconds and spun at 1,100×g for 5 seconds. The supernatant was removed without disrupting the pellet at the bottom of tube. The pellet was washed with another 200 μL of cold acetonitrile. The supernatant was again removed, and the tube was left open at room temperature until the pellet was almost dry. Finally, the pellet was dissolved in 0.1% TFA in deionized water.

### Sample Desalting and Peptide Isolation Prior to NanoLC-MS/MS Analysis

(1) Using Ziptip® with 0.6 μL C18 Resin

Human serum (or plasma) filtrate contains a large amount of salt, which has to be removed before the sample is analyzed by LC-MS/MS. First, 200 μL human serum (or plasma) filtrate sample was concentrated with a speed-vac to 10 μL and then acidified with 0.5% TFA in deionized water. A Ziptip® with 0.6 μL C18 resin was used to carry out the necessary desalting according to the manufacturer's instructions. The analyte was then concentrated in a speed-vac and resuspended to a final volume of 20 μL of 0.1% FA in deionized water.

(2) Using Reverse Phase Chromatography

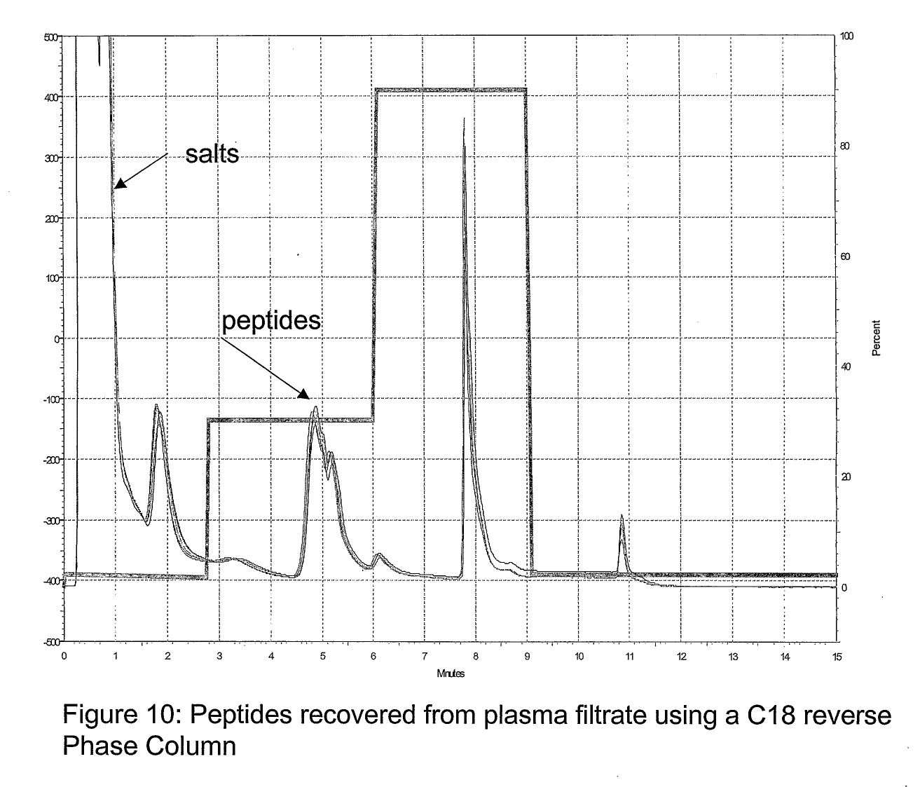

The filtrate was desalted using a Supelco 4.6mm×20 mm C18 wide pore 3μ column. Eighty percent of the filtrate was loaded onto the column using a Shimadzu HPLC and a buffer system consisting of mobile phase A (0.08% trifluoro acetic acid (TFA) in water) and mobile phase B (0.07% TFA in 100% acetonitrile) and a flow rate of 1.5 ml/min. The column was first equilibrated with buffer A and salts were remove by washing the column with 100% buffer A for 3 min. This was followed by a 3 min step to 30% buffer B to elute the peptides and then a step to 90% for 3 min to remove very hydrophobic peptides and clean the column. FIG. 10 shows the peptides recovered in the 30% step.

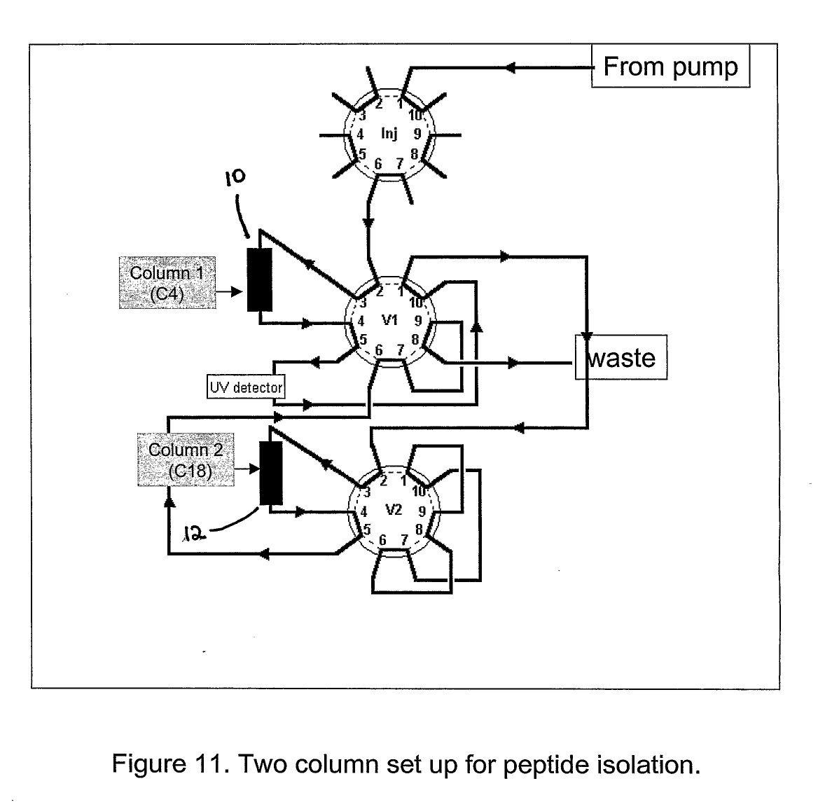

Alternatively, a two-column reverse phase method can be used as a way of separating peptides into different populations. For example, human serum (or plasma) filtrate could be cleaned and at the same time separated into two different peptide populations using a combination of two reverse phase polymeric media of different hydrophobicities. Referring to FIG. 11, the sample, e.g., filtrate, is first loaded onto a weak (C4) hydrophobic phase column 10 to bind comparatively large and hydrophobic peptides with molecular weight range between 1,000 Da and 12,000 Da. Salts and small (400-1000 Da) and/or hydrophilic peptides flow through and are captured by a second more hydrophobic (C18) column 12. Alternatively, the hydrophobicities of the columns can be adjusted to a different cut-off value, e.g., 3-4,000 Da.

The two columns are operated in tandem using multidimensional High Pressure Liquid Chromatography (HPLC) instrumentation that has the capability of having both columns online for sample loading and valve switching to independently elute each column and elute the two peptide population into two different fractions. The two columns are placed on line, equilibrated with buffer A and a sample is then loaded on column 1. Salts and small/hydrophilic peptides flowing through column 1 are captured by column 2. The salts are removed by washing both columns with 4 ml of buffer A. Column 2 (C18) is taken off line and column 1 (C4) is eluted using 3 ml of 25% buffer B, followed by 3 ml of 90% buffer B. Column 1 is taken off-line, and column 2 is then eluted with a step to 90% buffer A.

Using the reverse phase approach, the low molecular weight (LMW) peptides (approx. 400-12,000 Da) can also be isolated directly from serum/plasma and separated from the high molecular weight (HMW) proteins using the principle that the hydrophobicity of a biological molecule increases with increasing molecular weight. The binding behavior of peptides and proteins depends on the surface area and the hydrophobicity of the polymeric reversed phase chromatographic resin. The cut-off is dictated by the percentage of mobile phase used to elute the peptides. It has been found that 35% organic solvent is the upper limit. Concentrations of mobile phase higher than 35% organic result in elution of higher MW proteins. This method uses reverse phase polymeric media with large pores to enhance mass transport and to enable extremely rapid separations of fragments/peptides from high molecular weight proteins thus minimizing the risk of endogenous proteolytic degradation. Separation of the fragments/peptides can be accomplished in less than 10 min. Plasma and or serum is diluted with 100 μl of 1×PBS, 7.2 M guanidine pH 7.4 and a final concentration of 5 mM reducing agent (either 5 mM DTT, or 5 mM TCEP) to break biomolecular interactions, and the sample is immediately injected onto the reversed phase column. HMW proteins and LMW peptides are bound to the first column and eluted with a step to between 25%-30% buffer B and 90% buffer B respectively, as described above. Salts and other non-bound species are washed off the resin by running between 5-10 column volumes of the acidic aqueous buffer through the column. The 25-30% acetonitrile eluted fraction is concentrated to 20 μl using a speed vac. Ten pl of the concentrated fraction is further separated by on-line LCMS 1-D reverse phase chromatography nano-LCMS on a linear ion trap, as described herein.

### NanoLC-MS/MS Analysis

NanoLC-MS/MS analysis was performed on an UltiMate™ Nano HPLC system (LC Packings-Dionex, Marlton, N.J.) and LTQ-FT™ mass spectrometer (Thermo Electron, San Jose, Calif.). For the sample injection, FAMOS™ well plate Microautosampler (LC Packings-Dionex, Marlton, N.J.) was used. The capillary column used for LC-MS/MS analysis (150×0.075 mm) was obtained from New Objective (Woburn, Mass.) and slurry packed in house with 5 μm, 200 Å pore size Magic C18 stationary phase (Michrom Bioresources, Auburn, Calif.). The mobile phase A for the LC separation was 0.1% formic acid in deionized water and the mobile phase B was 0.1% formic acid in acetonitrile. Half of the volume (10 μL) of filtrate obtained from the Ziptip desalting step was loaded on to the LC column. The electrospray ionization voltage was set at 1.8 kV and the normalized collision energy was set at 28% for MS/MS. The temperature of the ion transfer tube was 245° C. The flow rate was maintained at 200 nL/min after splitting. Data dependent ion selection was used to select the 8 most abundant ions from a MS scan for MS/MS analysis with LTQ FT system. A precursor ion was excluded from further LTQ MS/MS analysis for 1 min.

The chromatography gradient was set up to give a linear increase from 2% B to 40% B in 35 minutes and from 40% B to 60% B in 20 minutes and from 60% B to 80% B in 5 minutes. The dynamic exclusion list consisted of 200 precursor masses.

### Data Processing and Analysis

Protein/peptide identifications were obtained using the SEQUEST algorithm from Thermo Electron (San Jose, Calif.). The human database used was extracted from the nr database on Sep. 9, 2004 (ftp://ftp.ncbi.nih.gov/blast.db/FASTA/). Search parameters used were as follows: no enzyme, no static modification, peptide mass tolerance set to 1.5 amu, fragment ion mass tolerance set to 0.0, number results scored set to 250. In this study, for a peptide to be considered a potentially positive identification the acceptance criteria required that the Xcorr be over 1.80 for singly-charged ions, 2.50 for doubly-charged ions, and 3.50 for triply-charged ions, additionally the mass error must be less than 2 ppm.

### EXAMPLE I

### Analysis of the LMW Filtrate

Five hundred μL human serum was fractionated by ultrafiltration as described without being subjected first to enzymatic digestion, and the filtrate, depleted of high molecular weight (HMW) proteins, was collected. A commercial protein assay kit (BCA) was used to determine the protein concentrations of the starting material and the filtrate. The values were about 60,000 μg/mL and 190 μg/mL, respectively. A yield of ˜0.33 mg of the low molecular weight (LMW) fraction was obtained from ˜30 mg of proteinaceous material contained in a human serum sample (relative to a standard, bovine serum albumin). This amount of material represents approximately 1% of the initial protein mass and demonstrates the effectiveness of the ultrafiltration approach as a depletion method.

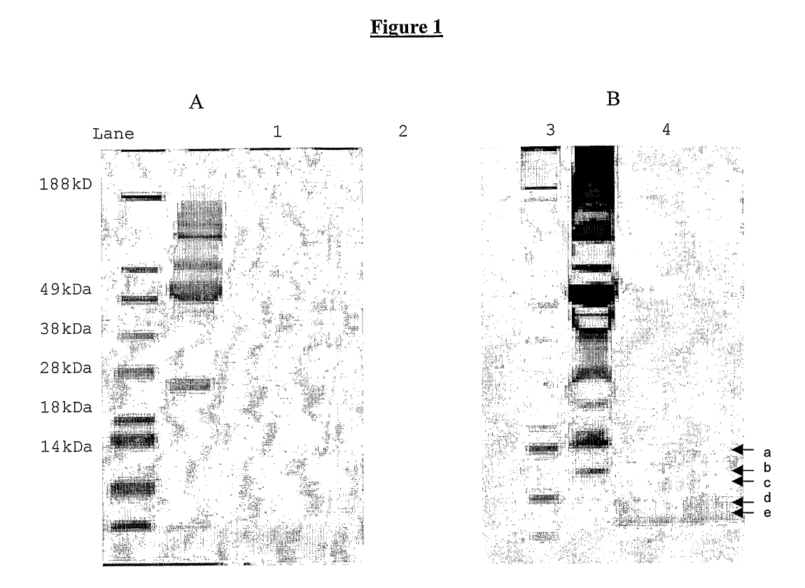

SDS PAGE analysis of the serum filtrate, concentrated either by cold acetonitrile precipitation or by use of a speed-vac, showed two weak bands in the low LMW region on the Coomassie® blue stained gel (see arrows, lane 3 and 4 in FIG. 1A). In order to improve detection sensitivity, the gel was destained and subjected to silver staining. The effectiveness of the ultrafiltration approach to successfully remove the HMW protein fraction was demonstrated by the fact that only bands having a molecular weight less than 10,000 Da were observed (see lanes 3 and 4 of the silver stained gel, FIG. 1B). Following speed-vac concentration, the salt was removed by a desalting step on a C18 Ziptip® prior to LC-MS/MS analysis.

A concern with the use of an ultrafiltration step to remove HMW proteins from serum relates to the potential loss of peptides that are strongly bound to carrier proteins, such as albumin. Although the addition of an organic solvent (10% acetonitrile) was used to disrupt hydrophobic interactions before the filtration step in an effort to minimize such losses, SDS PAGE analysis suggested that there may indeed be some peptide losses if one compares the intensity of bands “a” to “e” in lanes 2 to 4 of FIG. 1B. In this situation, however, the amount of peptide loss appears to be relatively consistent as yield measurements and SDS PAGE analysis of replicate isolations were found to be reproducible.

### EXAMPLE II

### NanoLC-MS/MS Analysis

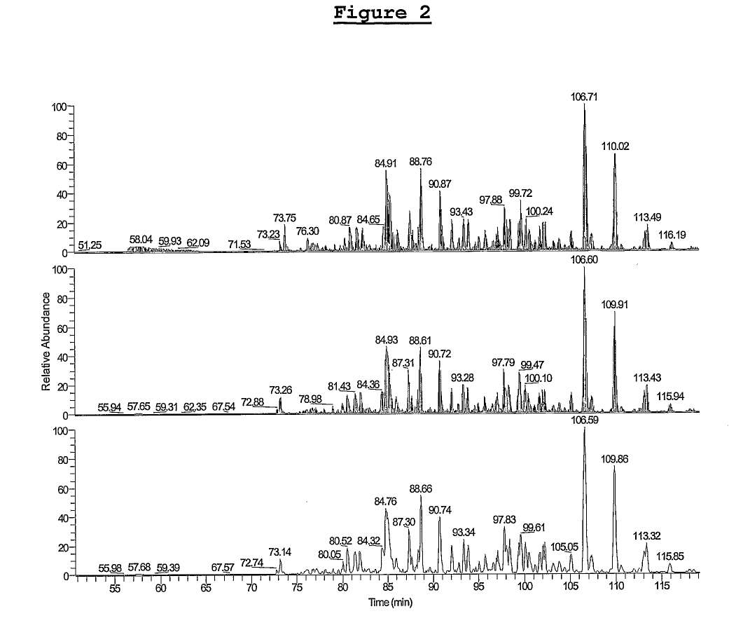

The LC-MS/MS analysis of a serum filtrate was performed at least three times for each sample. To assess reproducibility, the average coefficient of variation (CV) of the retention time was measured for five different peptides, and the value was found to be 0.15% for three runs. A typical comparison of the resulting total ion chromatograms is shown in FIG. 2. Peptides were identified by MS/MS fragmentation in a hybrid linear ion trap-Fourier transform mass spectrometer (LTQ-FTMS) with conservative values for sequence assignment (Xcorr(1+, 2+, 3+)=1.80, 2.50, 3.50).⁹ To get higher confidence peptide identifications, the hybrid FT-ICR instrument was used to acquire accurate mass measurements (within 2 ppm) during the survey MS scan (see, e.g., PCT Appl. No. PCT/US05/30713, which is hereby incorporated by reference herein) . Therefore, in addition to high Xcorr criteria for the assignment of peptide sequence, high mass accuracy obtained in the FTMS was used as another filter to assure accuracy of the assignment.

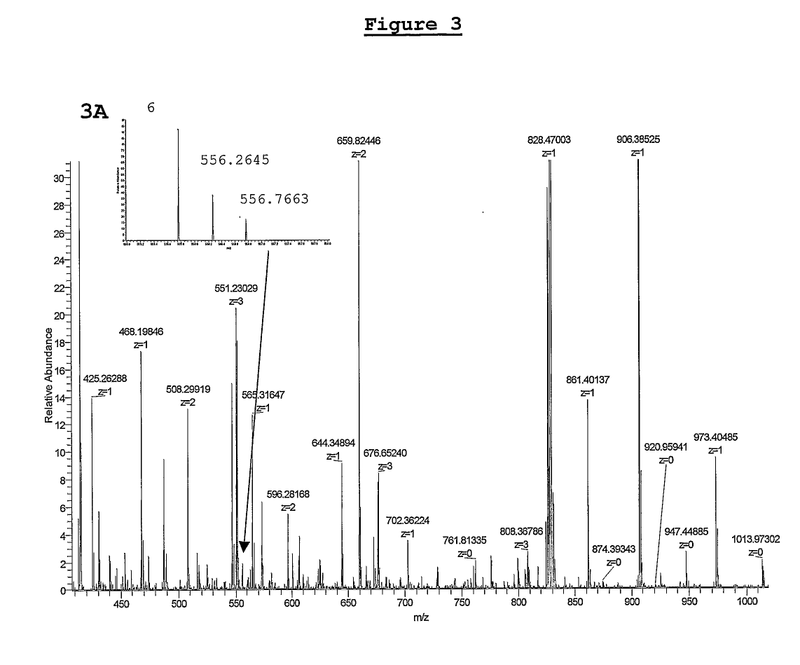

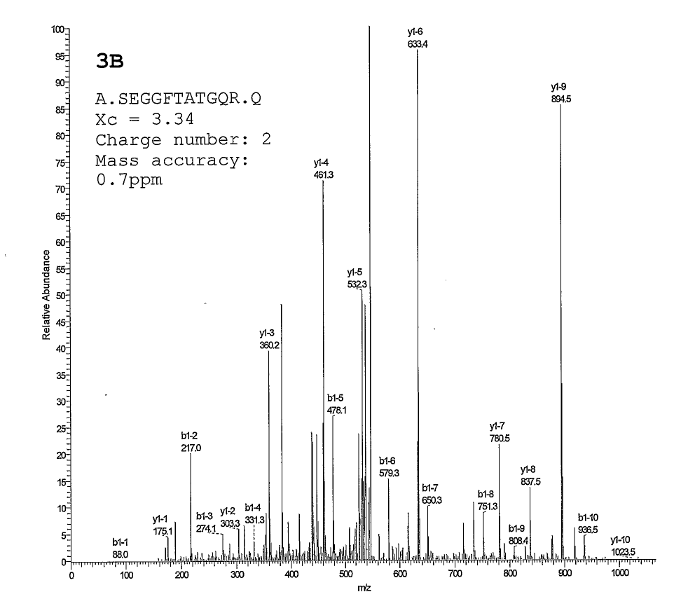

FIG. 3 shows an example of the type of data observed for the peptide SEGGFTAATGQR (presumed progenitor protein—extracellular matrix protein 1), which was identified as a doubly charged ion with an Xcorr of 3.34 and mass accuracy of 0.7 ppm. The extra filtering of data is particularly important in peptidome applications where many of the peptides are singly charged. Most of the proteins were identified from a single peptide (see FIG. 3A for typical results), and the decision not to use a trypsin digestion step precludes the use of trypsin specificity as an additional search criterion. In addition, the availability of an exact mass measurement greatly narrows down the number of possible candidates in a database search, which is particularly important for candidate sequences that contain several isobaric residues.

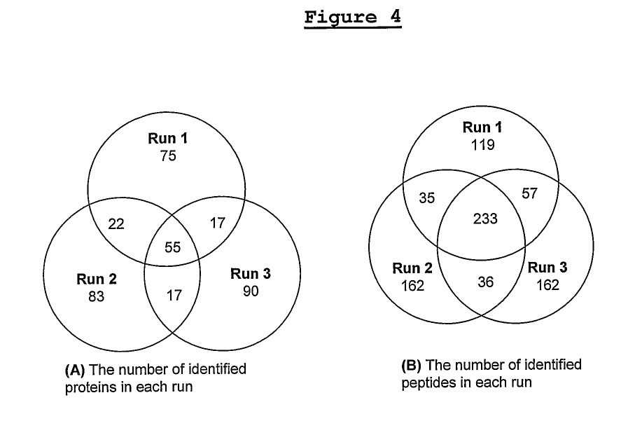

It has been noted previously that low level peptides may not be detected in a LC-MS/MS analysis due to the time constraints of the measurement, and, thus, repeated analyses are required.¹⁰ In addition, many correctly identified peptides, and particularly the lower abundance fragments, would also not be expected to be observed in replicate analyses because of the limitations of LC/MS. However, with the use of the LTQ-FT system, a total of 804 unique peptides, representing 359 unique proteins, were identified in human serum filtrate. Of these, 233 unique peptides and 55 proteins were identified in all of the triplicate runs (see FIG. 4). Table 2 lists 61 progenitor proteins and their 337 unique peptides, which were identified by MS/MS sequencing in at least two different runs and were measured with 2 ppm or better mass accuracy.

TABLE 2

| | | Number of |
|---|---|---|
| | | Unique |
| ID | Protein Name | Peptides |
| FIBA | Fibrinogen alpha/alpha-E chain | 118 |
| ITH4 | Inter-alpha-trypsin inhibitor heavy chain | 38 |
| CO3 | Complement C3 | 18 |
| CO4 | Complement C4 | 22 |
| APE | Apolipoprotein E | 16 |
| KNG | Kininogen | 15 |
| TYB4 | Thymosin beta-4 | 14 |
| APA4 | Apolipoprotein A-IV | 11 |
| HEP2 | Heparin cofactor II | 7 |
| F13A | Coagulation factor XIII A chain | 6 |
| THRB | Prothrombin | 6 |
| APC3 | Apolipoprotein C-III | 5 |
| APA1 | Apolipoprotein A-I | 4 |
| FIBB | Fibrinogen beta Chain | 4 |
| APL | Apolipoprotein L | 3 |
| A1AT | Alpha-1-antitrypsin | 3 |
| A2HS | Alpha-2-HS-glycoprotein | 2 |
| CO2 | Complement C2 | 2 |
| MYH4 | Myosin heavy chain, skeletal muscle, fetal | 2 |
| CA14 | Collagen alpha 1(IV) chain | 2 |
| ECM1 | Extracellular matrix protein 1 | 2 |
| ITAD | Integrin alpha-D | 2 |
| LOX5 | Arachidonate 5-lipoxygenase | 2 |
| NPL1 | Nucleosome assembly protein 1-like 1 | 2 |
| PLE1 | Plectin 1 | 2 |
| TTC3 | Tetratricopeptide repeat protein 3 | 2 |
| A2A1 | Adapter-related protein complex 2 alpha 1 | 1 |
| A2AP | Alpha-2-antiplasmin precursor | 1 |
| APF | Apolipoprotein F | 1 |
| ARVC | Armadillo repeat protein deleted in velo | 1 |
| ARY1 | Arylamine N-acetyltransferase 1 | 1 |
| BASP | Brain acid soluble protein 1 | 1 |
| BAI3 | Brain-specific angiogenesis inhibitor 3 | 1 |
| CA12 | Collagen alpha 1(II) | 1 |
| CA16 | Collagen alpha 1(VI) Chain | 1 |
| CA1B | Collagen alpha 1(XI) chain | 1 |
| C1QB | Complement C1Q subcomponent, B chain | 1 |
| TP3B | DNA Topoisonerase III beta-1 | 1 |
| EPB2 | Ephrin type-B receptor 2 | 1 |
| GELS | Gelsolin | 1 |
| HLP1 | HAP1-like protein 1 | 1 |
| HRG | Histidine-rich glycoprotein | 1 |
| IBP5 | Insulin-like growth factor binding protein 5 | 1 |
| IRK4 | Inward rectifier potassium channel 4 | 1 |
| NEB1 | Neurabin-I | 1 |
| OSF1 | Osteoclast stimulating factor | 1 |
| P11A | Phosphatidylinositol 3-kinase catalytic SU | 1 |
| PLX4 | Plexin A3 | 1 |
| BM1 | Polycomb complex protein BMI-1 | 1 |
| C215 | Protein C21ORF5 | 1 |
| MEFV | Pyrin (marenostrin) | 1 |
| G3BP | Ras-GTPASE-activating protein binding protein | 1 |
| RUN2 | Runt-related transcription factor 2 | 1 |
| ERR2 | Steroid hormone receptor ERR2 | 1 |
| T2AZ | Stoned B-tflla-alpha and beta like factor | 1 |
| SYT1 | Synaptotagmin I | 1 |
| TYB0 | Thymosin beta-10 | 1 |
| VIME | Vimentin | 1 |
| VTDB | Vitamin D-binding protein | 1 |
| VWF | Von willebrand factor | 1 |
| Z151 | Zinc finger protein 151 | 1 |

Proteins identified from human serum according to the method of the invention using an LTQ-FT analyzer and the NCBI database. All proteins were observed in at least two runs and have both an Xcorr(1+, 2+, 3+) ≧ 1.8, 2.5, 3.5 and at least 2 ppm mass accuracy.

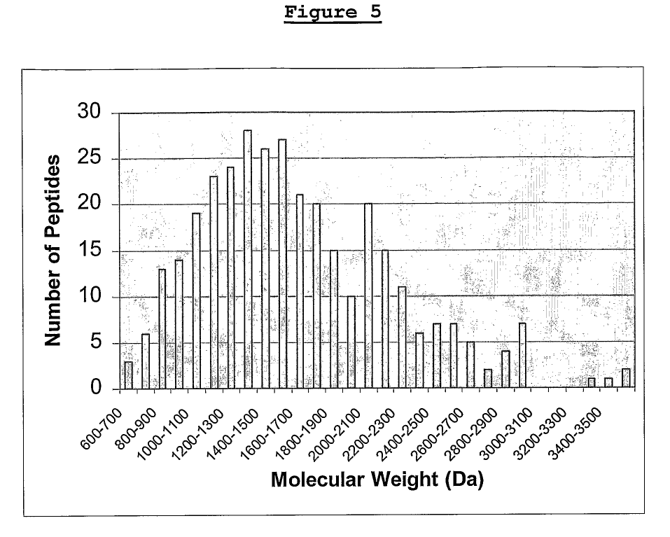

As is consistent with the SDS-PAGE results, albumin or albumin related fragments were not observed in the LC/MS analysis. FIG. 5 shows that the major proportion of the peptides identified with a high probability assignment had a MW range of approximately 1000 to 2300, which is a range that is not easily detected in 2D gel electrophoresis.

### EXAMPLE III

### Observance of Peptide Laddering

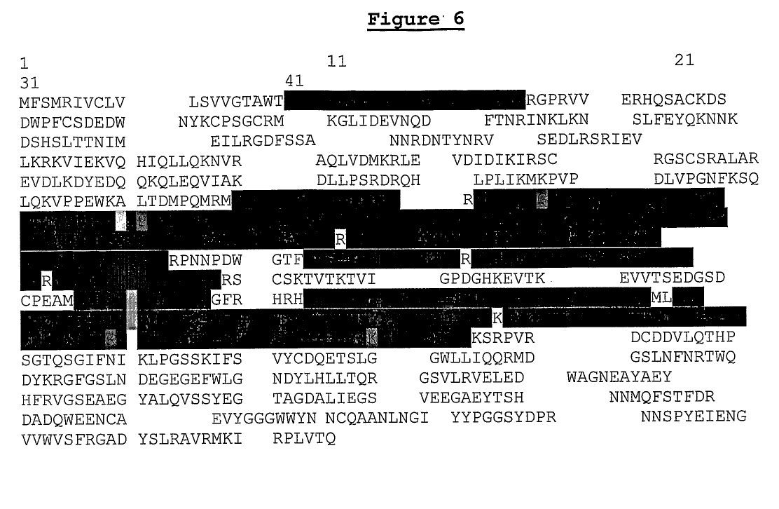

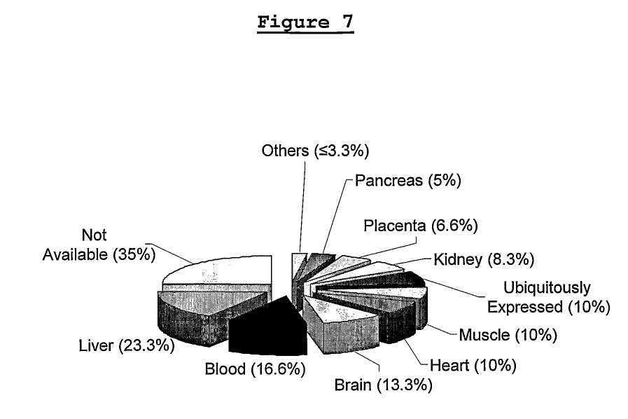

Many identified peptides were derived from the same protein sequence, a phenomenon referred to here as peptide laddering. These ladders are the result of progressive N- and C-terminal amino acid cleavages. In total, 34 peptide ladders were observed, derived from 17 different serum proteins with cleavage at diverse residues such as lysine, proline and glycine (Tables 3 and 4, and FIG. 6).

TABLE 3

| | | |
|---|---|---|
| | | No. of peptides in |
| ID | Peptide ladders identified in human serum. | ladder |

| | | |
|---|---|---|
| FIBA ^{a} | T.ADSGEGDFLAEGGGVR.G | 13 |
| | R.GGSTSYGTGSETESPRNPSSAGSWNSGSSGPGSTGN | 33 |
| | R | |
| | NPGSSGTGGTATWKPGSSGPGPGSTGSWNSGSSGTGS | |
| | TGNQNPGSPRPGSTGTWNPGSSE.R | |
| | R.GSAGHWTSESSVSGSTGQWHSESGSFRPDSPGSGNA | 5 |
| | .R | |
| | R.REYHTEKLVTSKGDKEL.R | 4 |
| | R.HPDEAAFFDTASTGKTFPGFFSP.M | 4 |
| | L.GEFVSETESRGSESGIFTNTKESSSHHPGIAEFPSRG.K | 8 |
| | K.SSSYSKQFTSSTSYNRGDSTFESKSYKMADEAGS | 34 |
| | EADHEGTHSTKRGHA.K | |
| ITH4 ^{a} | R.MNFRPGVLSSRQLGLPGPPDVPDHAAYHPF.R | 30 |
| CO3 ^{a} | R.SSKITHRIHWESASLLRSEETKENEGFTVTAEGK.G | 16 |
| CO4 ^{a} | R.TLEIPGNSDPNMIPDGDFNSYVR.V | 4 |
| | R.NGFKSHALQLNNRQIRGLEEELQFSLGSKINVKVG | 12 |
| | GNSKGTLKVL.R | |
| | K.DDPDAPLQPVTPLQLFEG.R | 4 |
| KNG ^{a} | R.GHGLGHGHEQQHGLGHGHKF.K | 3 |
| | F.KLDDDLEHQGGHVLDHGH.K | 7 |
| | K.RPPGFSPFR.S | 2 |
| | K.DDPDAPLQPVTPLQLFEG.R | 4 |
| APE ^{a} | A.KVEQAVETEPEPELRQQ.T | 4 |
| | A.TVGSLAGQPLQERAQAWGERL.R | 4 |
| | G.LVEKVQAAVGTSAAPVPSDNH.- | 6 |
| TYB4 ^{a} | K.KTETQEKNPLPSKETIEQEKQAGES.- | 14 |
| HEP2 ^{a} | G.GSKGPLDQLEKGGETAQSADPQWEQLNN.K | 7 |
| APA4 ^{a} | K.GNTEGLQKSLAELGGHLDQQVEEFR.R | 3 |
| | R.LAPLAEDVRGNL.K | 5 |
| F13A ^{a} | R.AVPPNNSNAAEDDLPTVELQGVVPR.G | 5 |
| THRB ^{a} | KSLEDKTERELLESYIDG.R | 5 |
| | R.TATSEYQTFFNPR.T | 4 |
| FIBB ^{a} | R.EEAPSLRPAPPPISGGGY.R | 3 |
| | N.DNEEGFFS.A | 2 |
| APC3 ^{a} | K.TAKDALSSVQESQVAQQA.R | 2 |
| A2HS ^{a} | R.HTFMGVVSLGSPSGEVSHPRKT.R | 4 |
| APL ^{a} | A.EEAGARVQQNVPSGTDTGDPQSKPLG.D | 2 |
| A1AT ^{a} | A.EDPQGDAAQKTDTSH.H | 2 |
| CO2 ^{a} | F.SHMLGATNPTQKTKESLG.R | 2 |

^{a} FIBA: Fibrinogen α/α-E CHAIN; ITH4: Inter-α-trypsin inhibitor; CO3: Complement C3; CO4: Complement C4; APE: Apolipoprotein E; TYB4: thymosin β-4; KNG: Kininogen; HEP2: Heparin cofactor II; F13A: Coagulation factor XIII A; APA4: Apolipoprotein A-IV; THRB: Prothrombin; FIBB: Fibrinogen β; A2HS: α-2-HS-Glycoprotein; APC3: Apolipoprotein C-III; APL: Apolipoprotein L; CO2: Complement C2; A1AT: α-1-antitrypsin.

TABLE 4

N-terminal ladders of fibrinopeptide A (FPA).

| | | |
|---|---|---|
| No. | MH+ | Peptide Sequences |

| | | |
|---|---|---|
| 1 | 1536.69 | T.ADSGEGDFLAEGGGVR.G |
| 2 | 1465.66 | A.DSGEGDFLAEGGGVR.G |
| 3 | 1350.63 | D.SGEGDFLAEGGGVR.G |
| 4 | 1263.60 | S.GEGDFLAEGGGVR.G |
| 5 | 1077.53 | G.EGDFLAEGGGVR.G |
| 6 | 1380.59 | T.ADSGEGDFLAEGGGV.R |
| 7 | 1309.55 | A.DSGEGDFLAEGGGV.R |
| 8 | 1194.53 | D.SGEGDFLAEGGGV.R |
| 9 | 1107.50 | S.GEGDFLAEGGGV.R |
| 10 | 1050.47 | G.EGDFLAEGGGV.R |
| 11 | 921.43 | E.GDFLAEGGGV.R |
| 12 | 864.41 | G.DFLAEGGGV.R |
| 13 | 749.38 | D.FLAEGGGV.R |

These results suggest that proteases such as thrombin might be involved in the generation of the serum peptidome. Table 3 also shows that the number of peptides in a ladder is variable; the ladders observed ranged from 4 to 34 members. The most diverse ladder was derived from the fibrinogen a/a-E chain with numerous peptides that were identified from different parts of the protein. The location of the peptide fragments in the fibrinogen structure is shown by the shaded regions in FIG. 6. In addition, the shaded boxes in this figure mark the predominant cleavage sites, as measured by the frequent observation of certain high abundance peptides in the MS/MS analysis. As an example, Table 4 shows two ladders generated from residues 20 to 33 of fibrinogen (box 1 in Table 3) comprising 5 and 8 peptide members, respectively. The ladders were generated by a single processing of a C-terminal glycine residue as well as by multiple steps of N-terminal processing; the significance of this peptide will be discussed below.

It is to be expected that serum would contain a plethora of these ladder peptides, which could be generated from the enzymes activated during the clotting process. This expectation has resulted in the preference for plasma over serum in many proteomic studies. Certainly, the diversity of proteins with peptide ladders shown in Tables 3 and 4 supports this view, but it must be recognized that currently most clinical specimen banks consist of serum samples. However, the specificity of the laddering process may contain useful information when correlated with the presence of specific peptides according to the methods of the invention as many diseases are associated with coagulation defects.^{14,15} Interestingly, several of the cleaved proteins are not known to be involved in the clotting process, such as inter-α-trypsin inhibitor, apolipoprotein E and A-IV, thymosin-β-4 and kininogen. Furthermore, if non-specific proteolysis were a general phenomenon, it would be expected that all major serum proteins would exhibit the laddering process. In this study, however, cleavage products of the following abundant proteins were not observed: human serum albumin, immunoglobulins, transferrin, alpha-2-macroglobulin, haptoglobin, lipoprotein a, alpha-1-acid glycoprotein, factor H, ceruloplasmin, complement factor B, prealbumin, C9 complement, Clq complement and C8 complement.

One peptide of diagnostic significance, Fibrinopeptide A (FPA, T.ADSGEGDFLAEGGGVR.G; the residues on either side of the period refer to the first residue of the adjacent peptide), which was observed in this peptidome study (see FIGS. 4 and 6), is known to be released as part of the clotting process. An elevated level of this peptide may indicate an abnormal clotting process, such as disseminated intravascular coagulation, cellulites, ovarian cancer¹⁴ and systemic lupus erythematosus.¹⁵ Measurement of FPA is used in clinical chemistry to diagnose certain types of leukemia, which are associated with disseminated intravascular coagulation. The results described here suggest that the clinical measurement could be complicated, however, by multi-step proteolysis. Our data show that after the generation of the parent peptide, the C-terminal arginine of FPA was enzymatically cleaved by an unknown enzyme to generate the peptide (T.ADSGEGDFLAEGGGV.R) lacking the C-terminal glycine residue. The peptide ladder is generated by digestion of the FPA fragment by an N-terminal exopeptidase(s) to give the products shown in Table 3.

Another example of a peptide of potential diagnostic significance that exhibits laddering is shown in Table 2 for the sequence (R.SSKITHRIHWESASLLRSEETKENEGFTVTAEGK.G) and is derived from complement C3. One of the daughter peptides (R.SSKITHRIHWESASLLR.S) is known as the C3f fragment and is thought to be related to inflammation.¹⁶ It was reported that C3f fragments were produced less abundantly in chronic asthma mice than in control mice.¹⁷ The flanking arginine residue from both the N-terminal and the C-terminus of this peptide (R.SSKITHRIHWESASLLR.S) has been reported to be cleaved by factor I.¹⁸ Also Factor I may be responsible for the generation of peptide (R.EGVQKEDIPPADLSDQVPDTESET.R), part of C3g fragment by cleavage of the arginine residue flanking the C-terminus.¹⁹

To resolve the issues around the use of plasma or serum, the original serum study was repeated on a matched set of samples obtained from the same 36 year old healthy male. A total of 87 unique peptides from 34 proteins were identified from the plasma filtrate and 117 unique peptides from 50 proteins were identified from the serum filtrate. Table 5 shows that many of the abundant peptide ladders seen in serum were also present in plasma, for example fibrinogen was observed with a total of 37 peptides in both samples. While plasma collection processes can be variable, these studies do not suggest that there is a clear preference for plasma over serum in proteomic analyses. It should be noted that the number of unique peptides identified from the matched plasma and serum filtrate samples was lower than that of Sigma serum filtrate. This could be explained by the extra complexity of the commercial Sigma serum sample, which was prepared by the pooling of samples from multiple donors.

TABLE 5
Comparison of abundant proteins present in a set of plasma
and serum samples from the same individual.

| | | Serum | | | |
|---|---|---|---|---|---|
| Protein | | | Num. of | Plasma | |
| ID | Protein Name | Rank | peptides | Rank | Num. of peptides |
| FIBA | Fibrinogen alpha- | 1 | 37 | 1 | 37 |
| | alpha/E chain | | | | |
| CO3 | Complement C3 | 2 | 12 | 2 | 8 |
| CO4 | Complement C4 | 3 | 11 | 3 | 6 |
| TYB4 | Thymosin beta-4 | 5 | 10 | 6 | 2 |
| APOL1 | Apolipoprotein-L 1 | 12 | 2 | 4 | 2 |
| TTHY | Transthyretin | 18 | 1 | 5 | 2 |
| FIBB | Fibrinogen beta | 23 | 1 | 9 | 1 |
| | chain | | | | |
| KNG | Kininogen | 25 | 1 | 7 | 1 |

All identified peptides in the LC-MS/MS study had a molecular weight less than 4,000 Da if ZipTip C18 was used to clean the sample (see FIG. 5), which was different from the results of SDS PAGE analysis of unfractionated plasma (see FIG. 1B, lane 4). A possible reason for this result was that the use of a desalting and concentration step (ZipTip C18) before the MS analysis which could result in the loss of larger hydrophobic peptides and alternative sample preparation procedures are being evaluated to overcome such losses.

### REFERENCES

(1) Anderson, N. L.; Anderson, N. G. Mol. Cell. Proteomics. 2002, 1, 845-867.
(2) Tirumalai, R. S.; Chan K. C.; Prieto, D. A.; Issaq, H. J.;

Conrads T. P.; Veenstra T. D. Mol. Cell. Proteomics. 2003, 2, 1096-1103.
(3) Adkins, J. N.; Varnum, S. M.; Auberry, K. J.; Moore, R. J.; Angell, N. H., Smith, R. D.; Springer, D. L.; and Pounds, J. G. Mol. Cell. Proteomics 2002, 1, 947-955.
(4) Schrader, M.; Schulz-Knappe, P. Trends in biotechnol. 2001, 19, S55-S60.
(5) Richter, R.; Schulz-Knappe, P.; Schrader, M.; Standker, L.;

Jurgens, M.; Tammen, H.; Forssmann, W. G. J Chromatogr B Biomed Sci Appl. 1999, 726, 25-35.
(6) Zhou, M.; Lucas, D. A.; Chan, K. C.; Issaq, H. J.; Petricoin, E. F.; Liotta, L. A.; Veenstra, T. D.; Conrads, T. P. Electrophoresis 2004, 25, 1289-1298.
(7) Harper, R. G.; Workman, S. R.; Schuetzner, S.; Timperman, A. T.; Sutton, J. N Electrophoresis 2004, 1299-1306.
(8) Strader, M. B.; Verb erkmoes, N. C.; Tabb, D. L.; Connelly, H. M.; Barton, J. W.; Bruce, B. D.; Pelletier, D. A.; Davison, B. H.; Hettich, R. L.; Larimer, F. W.; Hurst, G. B. J. Proteome Res. 2004, 3, 965-978.
(9) Tabb, D. L.; McDonald, W. H.; Yates, J. R. III Journal of Proteome Research 2002, 1, 21-26.
(10) Shen, Y.; Jacobs, J. M.; Camp, D. G. II; Fang, R.; Moore, R. J.; Smith, R. D.; Xiao, W.; Davis, R. W.; Tompkins, R. G. Anal Chem. 2004, 76, 1134-1144.
(11) Cugno, M.; Scott, C. F.; Salerno, F.; Lorenzano, E.; Muller-Esterl, W.; Agostoni, A.; Colman, R. W. Thromb Haemost. 1999, 82, 1428-1432.
(12) Hannappel, E.; van Kampen, M. J Chromatogr. 1987, 397, 279-285.
(13) Smith, D. B.; Janmey, P. A.; Herbert, T. J.; Lind, S. E. J Lab Clin Med. 1987, 110, 189-195.
(14) Bergen, H. R. III; Vasmatzis, G.; Cliby, W. A.; Johnson, K. L.; Oberg, A. L.; Muddiman, D. C. Dis. Markers. 2003, 19, 239-249.
(15) Gando, S.; Nanzaki, S.; Sasaki, S.; Kemmotsu, O. Thromb. Haemost. 1998, 79, 1111-1115.
(16) De Bruijn, M. H. L.; Fey, G. H. Proc. Natl. Acad. Sci. U.S.A. 1985, 82, 708-712.
(17) Yeo, S.; Roh, G. S.; Kim, D. H.; Lee, J. M.; Seo, S. W.; Cho, J. W.; Kim, C. W.; Kwack, K. Proteomics 2004, 4, 3308-3317.
(18) Harrison, R. A.; Farries, T. C.; Northrop, F. D.; Lachmann, P. J.; Davis, A. E. Complement 1988, 5, 27-32.
(19) Chaplin, H.; Monroe, M. C.; Lachmann, P. J. Clin. Exp. Immunol. 1983, 51, 639-646.
(20) Boire, A.; Covic, L.; Agarwal, A.; Jacques, S.; Sherifi, S.; Kuliopulos, A. Cell. 2005, 120, 303-313.
(21) Berriz, G. F.; King, O. D.; Bryant, B.; Sander, C.; Roth, F. P. Bioinformatics 2003, 19, 2502-2504.
(22) Wu, S.-L., Jardine, I., Hancock, W. S., and Karger, B. L. Rapid Commun. Mass Spectrom 2004, 19, 2201-2207.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A method for detecting disease associated proteolysis comprising:
providing a biological sample from a mammal;
isolating from said sample all low molecular weight peptides having a molecular weight of not more than approximately 12,000 Daltons;
directly detecting and determining the amino acid sequences of said isolated peptides by ionization mass spectrometry; and
relating the determined sequence information to a reference standard.

2. The method of claim 1, wherein the sample is a bodily fluid selected from the group consisting of blood (e.g., plasma or serum), saliva, urine, nipple aspirate, ductal lavage, sweat or perspiration, tumor exudates, joint fluid (e.g. synovial fluid), inflammation fluid, tears, semen and vaginal secretions.

3. The method of claim 1, wherein the step of isolating comprises one or more methods selected from the group consisting of:
reverse phase chromatography;
ultrafiltration; and
bulk electrophoresis.

4. The method of claim 1, wherein the step of directly detecting is carried out by electrospray ionization mass spectrometry.

5. The method of claim 1, wherein the step of directly detecting is carried out using an ion trap mass spectrometer.

6. The method of claim 1, wherein the step of directly detecting is carried out using a hybrid ion trap mass spectrometer coupled to a quadrapole, time-of-flight mass spectrometer.

7. The method of claim 1, wherein the step of directly detecting is carried out using a reversed phase-high performance liquid chromatography system connected to a nanospray ionization hybrid ion trap-fourier transform mass spectrometer.

8. The method of claim 1, wherein the step of relating comprises one or more methods selected from the group consisting of:
identifying specific peptides having elevated levels compared to a reference normal;
correlating peptides with specific proteins; and
identifying protein fragmentation cleavage sites and relating that cleavage site to the activity of a specific endogenous propeolytic enzyme.

9. The method of claim 1, wherein, prior to said step of directly detecting, further fractionating said isolated peptides by removing all peptides having a molecular weight of less than approximately 3,000-4,000 Daltons.

10. A method of identifying a potential biomarker for a disease or pathological condition, said method comprising the steps of:
a) obtaining a plurality of bodily fluid samples from a plurality of donors, wherein said plurality of donors comprise healthy individuals and patients at high risk for and/or having said disease or pathological condition;
b) isolating from said plurality of bodily fluid samples all low molecular weight peptides having a molecular weight of not more than approximately 12,000 Daltons;
c) directly detecting and determining the amino acid sequences of said isolated peptides by mass spectrometry;
d) determining a profile of low molecular weight peptide abundance in each of said samples;
e) comparing said abundance profiles of said patients at high risk for and/or having said disease or pathological condition with said abundance profiles of said healthy individuals; and
f) identifying any low molecular weight peptides that are present in the abundance profiles of said patients at high risk for and/or having said disease or pathological condition but not in, or at a reduced level in, the abundance profiles of said healthy individuals,
wherein any low molecular weight peptide so identified is a potential biomarker for said disease or pathological condition.

11. The method of claim 10, further comprising repeating said method steps so as to identify a plurality of potential biomarkers for said disease or pathological condition.

12. The method of claim 11, wherein said plurality of potential biomarkers for said disease or pathological condition comprise a panel of potential biomarkers.

13. The method of claim 12, wherein said panel of potential biomarkers is a peptide ladder.

14. A method of separating small peptides from large peptides and proteins comprising:
providing a column comprising polymeric, silica, zirconia reversed phase chromatographic particles equilibrated to retain hydrophobic compounds and not to retain salts and other hydrophilic compounds;
applying a sample comprising a mixture of proteins and peptides to said column;
washing hydrophilic compounds through said column;
washing said column to remove mildly hydrophobic, small peptides from said column;
washing said column to remove larger, more hydrophobic peptides away from proteins from said column; and
washing said column to separate proteins from mildly and very hydrophobic peptides.

15. The method of claim 14, further comprising
providing a second column comprising a polymeric, silica, zirconia reversed phase chromatographic resin equilibrated to retain small, hydrophilic compounds eluted from said first column.
